(19) United States
(12) Patent Application Publication
(10) Pub. No.: US 2014/0228550 A1
(43) Pub. Date: Aug. 14, 2014

(54) Process for Obtaining Proteins from a Native Substance Mixture

(71) Applicants: Steffen Hruschka, Oelde (DE); Wladislawa Boszulak, Oelde (DE)

(72) Inventors: Steffen Hruschka, Oelde (DE); Wladislawa Boszulak, Oelde (DE)

(73) Assignee: GEA Mechanical Equipment GmbH, Oelde (DE)

(21) Appl. No.: 14/124,561

(22) PCT Filed: Jun. 6, 2012
(86) PCT No.: PCT/EP2012/060675

§ 371 Date: Apr. 15, 2014

(30) Foreign Application Priority Data
Jun. 7, 2011 (DE) 10 2011 050 905.4

(51) Int. Cl. C07K 1/14

(52) U.S. Cl. CPC C07K 1/145
USPC 530/422

(57) ABSTRACT

A process is provided for obtaining proteins from native substance mixtures, wherein a native substance mixture is first finely disintegrated and optionally processed to form a flowable slurry (I) by adding liquid. The process includes the following steps: (i) setting the pH value of the slurry (I) in an alkaline range; (ii) adding at least one water-soluble organic solvent after setting the pH value of the slurry according to step (i); and (iii) separating a protein phase (VI) from the slurry after step (ii)

### BACKGROUND AND SUMMARY OF THE INVENTION

The present invention relates to a process for recovering proteins from a natural product mixture.

DE 195 29 795 C2 discloses a process which allows the recovery of oils, fats or waxes. Here, an aqueous slurry is separated into solid and liquid constituents in a centrifuge. A proportion of 5-75% by weight, based on the liquids content of the slurry, of an organic solvent is added to the aqueous slurry. DE 195 29 795 C2 addresses the problem of isolating a clean oil phase, an aqueous phase and a solid phase which has been freed of oil from the aqueous slurry. This process has been found to be suitable in principle for the recovery of oils, waxes and fats.

Known processes for producing proteins are production of a protein isolate at an alkaline pH or production of a protein concentrate at an acidic pH, which are preferably employed in the case of hexane-extracted shredded material but cannot be applied, in conjunction with the process of DE 195 29 795 C2, to a protein/lecithin mixture without an energy-intensive drying step.

In the light of this background, it is an object of the invention to obtain a protein phase of high purity.

The invention achieves this object by providing a process for recovering proteins from natural product mixtures, in particular shredded leguminous plants or shredded rapeseed plants, in which the mixture is firstly finely comminuted and optionally (if not liquid enough) processed by addition of a liquid to form a flowable slurry. The process comprises at least the following steps:
(i) setting of a pH of the slurry in the alkaline range, i.e. to a pH of greater than 7.0;
(ii) addition of at least one water-soluble organic solvent after setting the alkaline pH in the alkaline range; and
(iii) separation of a protein phase from the slurry.

Adhering to the order of these steps is particularly advantageous.

Here, unlike in DE 195 29 795 C2, a pH of the slurry in the alkaline range is set before addition of the water-soluble organic solvent. As a result, the solubility of the proteins in the aqueous medium is increased, they are partially dissolved and, if they are not completely dissolved, are present in at least finely divided and voluminous form in the solution and not in compact form like the other solids. The presence of a protein/lecithin mixture interferes with complete solubility of the proteins. After setting of the pH, the organic water-soluble solvent is added, as a result of which oil, inter alia, is displaced from the partially dissolved protein suspension.

The process of the invention thus makes it possible to recover proteins having a high purity since, inter alia, the increase in the solubility of the proteins obviously also results in loosening of bonds to, for example, impurities composed of cellulose or husks or the like.

The process can be used for recovering proteins. In addition, it can particularly advantageously be combined with recovery of oil from the mixture, which oil can be separated off as a separate phase by addition of the solvent in step b.

Solids or undissolved sediment are preferably separated off in a separate step after step (ii), i.e. the partial dissolution of the proteins, and before the actual isolation of the protein phase and optionally the oil phase.

The pH in step (i) is preferably equal to or greater than pH=9. As a result of the shift of the pH into the alkaline range in step (i), particularly good dissolution or partial dissolution of proteins in the aqueous solution is achieved. Better separation of the protein phase from the remaining solids can be effective as a result. Particularly favorable conditions for partial dissolution of the proteins are obtained at a pH of greater than pH=9 and in particular at a pH of pH=10±0.5.

A short-chain aliphatic alcohol can be employed as water-soluble organic solvent in step (ii). This relates first and foremost to readily available alcohols such as methanol, ethanol or propanol which are available in large quantities.

Since the addition of the solvent is associated with a decrease in the solubility of the proteins, it is advantageous for the content of water-soluble organic solvent in the slurry after addition of the water-soluble alcoholic solvent in step (ii) to be less than 45% by volume, preferably <15% by volume. An increased concentration above 45% by volume of alcoholic solvent displaces any oil to be separated off into an intermediate phase between the protein phase and the aqueous phase. This makes isolation of the oil phase more difficult and leads to less good results than below 45% by volume. The proteins remain compact and mix with the solid phase.

Separation in a centrifugal field is particularly useful for separating off the solid phases. Removal of the solid phase can preferably be effected by means of a clarifying decanter.

Removal of the solids leaves a mixture of aqueous alcoholic solution and proteins in an essentially aqueous form and possibly an oil phase. The interest is now in isolating the valuable constituents, i.e. the protein phase and the oil phase. The isolation of at least the protein phase in step (iii) is preferably carried out by means of the step (iii)-1, precipitation of the protein phase by adjusting the pH. As a result, the mixture comprises a solid phase and one or two liquid phases which can be separated into an oil phase, a protein phase and an alcoholic-aqueous phase in a centrifugal field in a subsequent step (iii)-2. This can preferably be effected by use of a three-phase separator.

Precipitation of the protein phase is preferably brought about by lowering the pH to the isoelectric point. Here, inter alia, individual precipitated proteins can clump together, as a result of which they can be separated even better from the liquid phases.

To improve the purity of the protein phase, it can be washed in step (iv) after isolation by adjustment of the pH.

The protein obtained is a “natural product” and largely polyphenol-free.

The invention will be illustrated below with the aid of an example and reference to the accompanying drawing.

### BRIEF DESCRIPTION OF THE DRAWING

FIG. 1 shows an illustrative process flow diagram

### DETAILED DESCRIPTION OF THE DRAWING

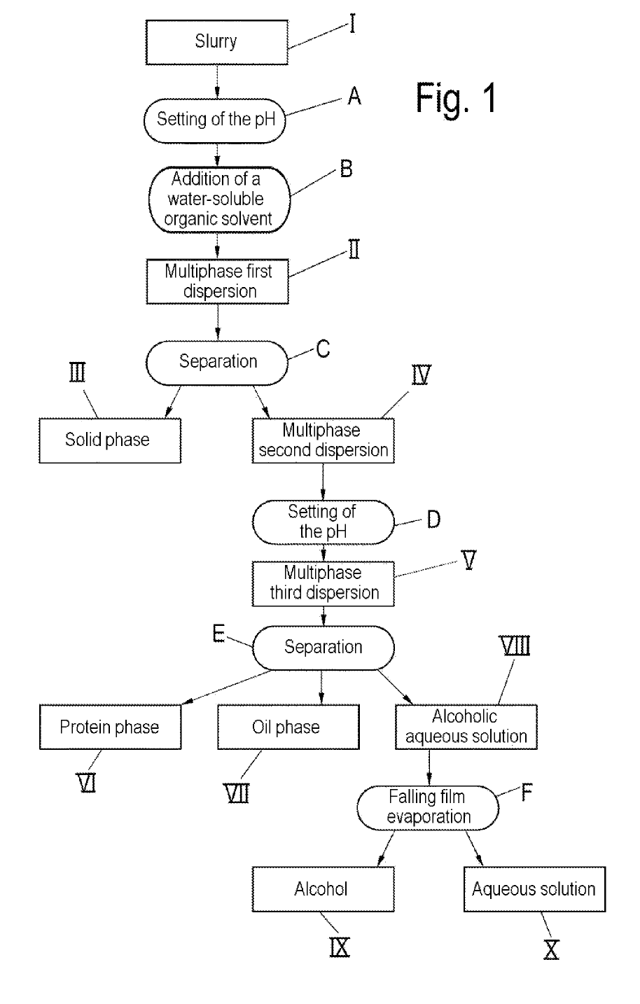

In the following, the process of the invention will be described in more detail with the aid of the specific sequence of steps shown in FIG. 1.

As starting material, use is made of a natural organic product mixture, preferably derived from legumes, rapeseed or micro-organisms. This mixture is firstly preferably comminuted and if appropriate converted by addition of water or another liquid, for example organic solvent, into a flowable slurry.

This slurry I can, for example, be produced from a press cake from oil recovery which has been suspended in water to form the slurry I. The slurry I is particularly preferably obtained from rapeseed or soybeans. The slurry I contains proteins in addition to oil constituents. Furthermore, the slurry I can also contain lecithin, polyphenols and solid constituents such as husks, the content of which should be very small both in the oil phase to be recovered and in the protein component to be recovered.

In a first process step A, the pH of the slurry I is shifted into the alkaline range by, for example, addition of sodium hydroxide solution. This increases the solubility of protein and the proteins are largely brought into solution. However, a small proportion of proteins can remain undissolved and finely dispersed in the aqueous slurry since the solubility of the proteins is limited by the proportion of oil and lecithin in the mixture. The pH of the dispersion after step (i) is preferably greater than pH=9, and the pH of the dispersion is particularly preferably pH=10.

In a second process step B, the alkaline dispersion is subsequently admixed with a short-chain aliphatic alcohol. This alcohol can preferably be selected from the group of alcohols consisting of methanol, ethanol and propanol. The addition of the alcohol results in a shift in the solubility equilibrium. A displacement extraction, in which the oil is displaced from the comminuted natural product matrix by the addition of alcohol, occurs.

Here, the alcoholic-aqueous alkaline dispersion II separates into a total of four phases, viz. an oil phase, an alcohol phase, a protein phase and a solid phase composed of husks and other solids. The volume of alcohol added in step (ii) should preferably be selected so that the alcohol content of the aqueous dispersion after step (ii) is less than 45% by volume. An alcohol content of <15% by volume has been found to be particularly useful in order to bring about a phase separation between the protein phase and the husk phase and obtain very pure individual phases. The bonds to other compounds, e.g. impurities composed of cellulose, for example of husks, are particularly preferably also weakened to such an extent that separation of the alcoholic-aqueous alkaline first dispersion II comprising the above-described plurality of phases occurs in a centrifugal field.

In process step C, after the addition of alcohol, a first separation in which the solid phase composed of husks and further constituents is removed from the multiphase first dispersion II is carried out. This process step C is carried out before the actual isolation of protein and allows the removal of undesirable solids. This removal of solids is preferably carried out as a centrifugal separation in a clarifying decanter.

After the separation, a pure solid fraction III and a multiphase second dispersion IV composed of at least one upper oil phase, an alcoholic-aqueous middle phase and a lower suspended protein phase is obtained.

In a subsequent process step D, or a step (iv), the proteins are precipitated by setting the pH in the region of the isoelectric point of the protein phase, resulting in a multiphase third dispersion V comprising a protein solid phase and two liquid phases, viz. an oil phase and an alcoholic-aqueous phase.

After precipitation of the proteins, a second separation is carried out in a process step E or a step (v). However, this time the protein phase, the oil phase and the alcoholic-aqueous phase are separated from one another. This is particularly preferably effected by centrifugal separation.

After process step E, a protein phase VI, an oil phase VII and an alcoholic-aqueous phase VIII are obtained in one or more steps.

In a further optional process step F, the alcohol IX can be recovered from the alcoholic-aqueous phase VII by falling film evaporation. An essentially aqueous solution X thus remains as residue from the process.

Examination of the products obtained (oil and proteins) has shown that improved separation behavior and, associated therewith, an even higher purity of the products, in particular the proteins isolated, could be achieved. Interfering impurities such as polyphenols and lecithin accumulate to a particularly high extent in the alcoholic-aqueous phase VIII, with polyphenols no longer being found or being found in only vanishingly small proportions in the protein phase which has been separated off and optionally washed.

In addition, it has been found that simultaneous addition of alkali and alcohol and reverse of the order of the steps (i) and (ii), leads to insufficient partial dissolution of the proteins occurring and thus to isolation of a protein phase freed of impurities occurring to only an unsatisfactory extent and a lower protein yield being obtained.

Specifically, the improved separation between protein phase and solids after addition of an alkali in step (i) or process step A is indicated by formation of a first dispersion II having a plurality of phases, as follows:
Uppermost phase: oil (yellow color)
Second phase: aqueous alcohol phase (turbid, brownish)
Third phase: protein phase comprising partially dissolved proteins (white-yellow phase)
Sediment: solid phase composed of husks and the like (black-green phase).

On varying the order of the steps (i) and (ii) or of process steps A and B, different purities of the solid phase were observed. Thus, in the case of the order of steps according to the process of the invention, the solid phase was greenish black and displayed few white protein interstices, i.e. only a low degree of marbling. The behavior was different when the order of steps was reversed, i.e. step (ii) before step (i), and with simultaneous addition of alcohol and alkali. Here, intensive marbling and thus undesirable mixing of the two phases, i.e. the protein phase and the solid phase, were observed.

After the centrifugal removal of the solids III according to process step C and lowering of the pH to the isoelectric point to precipitate the proteins according to step (iv) or process step D, the following phases are present in the multiphase third dispersion V:
Uppermost phase: oil (yellow color)
Second phase: aqueous alcohol phase (turbid, brownish)
Third phase: protein phase comprising precipitated proteins (white-yellow phase).

The proteins can, just like the oil, preferably be isolated in a subsequent centrifugal liquid-liquid-solid separation process in process step E. It was conspicuous here that a large part of lecithin and polyphenols which were hitherto found to a larger extent in the protein phase are now present to a greater extent in the alcoholic-aqueous phase, while the protein phase has a higher purity.

Furthermore, the use of protective gas can advantageously be dispensed with in the process.

Instead of the oil phase VI described, fats or waxes, for example, can also be separated off from the slurry in the same manner.

A rapeseed sample was processed by way of example using the process of the invention in order to recover proteins.

A rapeseed press cake of this type (100 g) consisted, according to analysis, of 90% by weight of dry matter, of which 31.77% was proteins, 18.31% was oils, 1.71% by weight was PP (polyphenol) and about 10% by weight was water.

The rapeseed material to be processed (100 g) was firstly finely comminuted by means of a shear head mixer with addition of 415 g of distilled water and processed to give a flowable slurry, and 10% strength alkali was then added to set a pH of the slurry in the region of 10 in the alkaline range (process step A).

The slurry was subsequently gently mixed for 30 minutes. 78.5 g of alcohol were then added as water-soluble organic solvent to this slurry after setting of the pH of the slurry.

The slurry was then centrifuged at 40° C. for two minutes and a protein phase which had settled in a glass beaker as lower layer above the cleanly separated off husks in a proportion by volume of 40% was separated off from the centrifugation fractions.

An amount of protein of 18.28 g (68.3%) could be separated off in this way and was largely polyphenol-free. The protein fraction was also very pure, in particular visibly free of husks and free of other visible impurities. This demonstrates a substantial advantage of adhering to the steps of pH adjustment, addition of the water-soluble organic solvent and then, either immediately or after further intermediate steps c), isolation of the protein phase, since the protein phase is particularly pure.

### TABLE OF REFERENCE NUMBERS

I Slurry
II Multiphase first dispersion
III Solid phase
IV Multiphase second dispersion
V Multiphase third dispersion
VI Protein phase
VII Oil phase
VIII Alcoholic aqueous solution
IX Alcohol
X Aqueous solution
Process step A setting of the pH
Process step B addition of a water-soluble organic solvent
Process step C separation
Process step D setting of the pH
Process step E separation
Process step F falling film evaporation

1-19. (canceled)

20. A process for recovering proteins from natural product mixtures, in which a natural product mixture is firstly finely comminuted and optionally processed by addition of a liquid to form a flowable slurry, the process comprising the steps of:
(i) setting of a pH of the slurry in the alkaline range;
(ii) adding at least one water-soluble organic solvent after setting the pH of the slurry; and
(iii) separating a protein phase from the slurry after adding the water-soluble solvent.

21. The process as claimed in claim 20, wherein an oil phase, a fat or a wax is separated off from the slurry after step (ii).

22. The process as claimed in claim 21, wherein the separation of the oil phase is carried out in one or more steps.

23. The process as claimed in claim 22, wherein the separation is carried out in a three-phase decanter or in at least two steps in two-phase decanters.

24. The process as claimed in 20, wherein a solid phase is separated off from the slurry before the protein phase is separated off.

25. The process as claimed in claim 20, wherein the pH in step (i) is greater than pH=7.

26. The process as claimed in claim 20, wherein the pH in step (i) is greater than pH=9.

27. The process as claimed in claim 20, wherein the pH in step (i) is pH=10±0.5.

28. The process as claimed in claim 20, wherein the setting of the alkaline pH of the slurry is effected by addition of an alkali.

29. The process as claimed in claim 28, wherein the alkali is a sodium hydroxide solution.

30. The process as claimed in claim 20, wherein the water-soluble organic solvent in step (ii) is a linear aliphatic alcohol.

31. The process as claimed in claim 30, wherein a content of water-soluble organic solvent in the slurry after addition of the alcohol in step (ii) is less than 45% by volume.

32. The process as claimed in claim 30, wherein a content of water-soluble organic solvent in the slurry after addition of the alcohol in step (ii) is less than 15% by volume.

33. The process as claimed in claim 22, wherein removal of the solid phase is effected in a centrifugal field.

34. The process as claimed in claim 22, wherein removal of the solid phase is effected by use of a clarifying decanter.

35. The process as claimed in claim 20, wherein isolation of at least the protein phase in step (iii) is carried out by the following steps:
(iv) precipitation of the protein phase by adjustment of the pH; and
(v) centrifugal separation of the protein phase, an alcoholic-aqueous phase and optionally an oil phase.

36. The process as claimed in claim 35, wherein the precipitation of the protein phase is effected by lowering the pH to an isoelectric point of the proteins.

37. The process as claimed in claim 35, wherein the protein phase is washed after separation in step (iii).

38. The process as claimed in claim 35, wherein recovery of alcohol from the alcoholic-aqueous phase is carried out after step (iii).

39. The process as claimed in claim 35, wherein recovery of alcohol is carried out by falling film evaporation.
